(19) United States
(12) Patent Application Publication
(10) Pub. No.: US 2025/0333404 A1
(43) Pub. Date: Oct. 30, 2025

(54) DEUTERATED COMPOUNDS

(71) Applicant: Neurocrine Biosciences, Inc., San Diego, CA (US)

(72) Inventor: Evan Smith, Carlsbad, CA (US)

(21) Appl. No.: 18/870,220

(22) PCT Filed: May 31, 2023
(86) PCT No.: PCT/US2023/067669

§ 371 Date: Nov. 27, 2024

Related U.S. Application Data
(60) Provisional application No. 63/348,122, filed on Jun. 2, 2022.

(51) Int. Cl. C07D 417/12; A61K 31/635; C07B 59/00

(52) U.S. Cl. CPC C07D 417/12; A61K 31/635; C07B 59/002

(57) ABSTRACT

The present disclosure provides deuterated compounds and their use as sodium channel blockers in the treatment or prevention of various diseases or disorders associated with sodium channels.

### TECHNICAL FIELD

The present invention relates to deuterated compounds and their use as sodium channel blockers in the treatment or prevention of various diseases or disorders associated with sodium channels.

### BACKGROUND

Voltage gated sodium channels (Nav's) are critical determinants of cellular excitability in muscle and nerve (see e.g., Hille, B, Ion Channels of Excitable Membranes (2001), Sunderland, M A, Sinauer Associates, Inc.). Four isoforms in particular, Nav1.1, Nav1.2, Nav1.3, and Nav1.6, account for the majority of sodium current in the neurons of the central nervous system. Nav1.3 is primarily expressed embryonically. Beyond the neonatal stage, Nav1.1, Nav1.2, and Nav1.6 are the critical isoforms that regulate neuronal signaling in the brain (see e.g., Catterall, W. A., Annual Review of Pharmacology and Toxicology (2014), Vol. 54, pp. 317-338).

Epilepsy is a condition characterized by excessive synchronous excitability in the brain that arises when the delicate balance of excitatory and inhibitory signals in the brain fall out of equilibrium. This can happen either due to an excess of excitation, or a deficiency of inhibition. Mutations in the genes encoding Nav channels have been linked to both types of disequilibrium.

### SUMMARY

The present application provides, inter alia, a compound of Formula I.

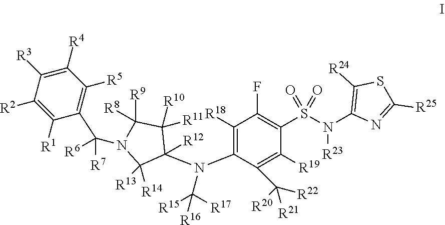

or a pharmaceutically acceptable salt thereof, wherein the constituent members are defined herein.

The present invention further provides pharmaceutical compositions comprising a compound described herein, or a pharmaceutically acceptable salt thereof, and a pharmaceutically acceptable carrier.

The present invention further provides methods of blocking a sodium channel (e.g., Nav1.6), comprising contacting the sodium channel with a compound described herein, or a pharmaceutically acceptable salt thereof.

The present invention further provides methods of blocking a sodium channel (e.g., Nav1.6) in a patient, comprising administering to the patient a compound described herein, or a pharmaceutically acceptable salt thereof.

The present invention further provides methods of treating a disease or disorder associated with a sodium channel in a patient, comprising administering to the patient a compound described herein, or a pharmaceutically acceptable salt thereof.

The present invention further provides compounds described herein, or a pharmaceutically acceptable salt thereof, for use in any of the methods described herein.

The present invention further provides uses of a compound described herein, or a pharmaceutically acceptable salt thereof, for the preparation of a medicament for use in any of the methods described herein.

### DESCRIPTION OF DRAWINGS

FIG. 1 shows a summary of observed metabolites of Compound 1 in mouse, rat, dog, monkey, and human hepatocytes.

FIG. 2 shows a summary of observed metabolites of Compound 1 in mouse hepatocytes and related information on LC-MS^{E} scans.

FIG. 3 shows a summary of observed metabolites of Compound 1 in rat hepatocytes and related information on LC-MS^{E} scans.

FIG. 4 shows a summary of observed metabolites of Compound 1 in dog hepatocytes and related information on LC-MS^{E} scans.

FIG. 5 shows a summary of observed metabolites of Compound 1 in monkey hepatocytes and related information on LC-MS^{E} scans.

FIG. 6 shows a summary of observed metabolites of Compound 1 in human hepatocytes and related information on LC-MS^{E} scans.

FIG. 7 shows proposed metabolic pathways of Compound 1 in mouse, rat, dog, monkey, and human hepatocytes.

FIGS. 8A-8B show LC-UV (FIG. 8A, λ: 260 nm-320 nm) and LC-MS (FIG. 8B) chromatograms of Compound 1 and its metabolites in mouse hepatocytes.

FIGS. 9A-9B show LC-UV (FIG. 9A, λ: 260 nm-320 nm) and LC-MS (FIG. 9B) chromatograms of Compound 1 and its metabolites in rat hepatocytes.

FIGS. 10A-10B show LC-UV (FIG. 10A, λ: 260 nm-320 nm) and LC-MS (FIG. 10B) chromatograms of Compound 1 and its metabolites in dog hepatocytes.

FIGS. 11A-11B show LC-UV (FIG. 11A, λ: 260 nm-320 nm) and LC-MS (FIG. 111B) chromatograms of Compound 1 and its metabolites in monkey hepatocytes.

FIGS. 12A-12B show LC-UV (FIG. 12A, λ: 260 nm-320 nm) and LC-MS (FIG. 12B) chromatograms of Compound 1 and its metabolites in human hepatocytes.

FIGS. 13A-13B show UV (FIG. 13A) and CID MS² (FIG. 13B) spectrum of Compound 1 (m/z 461.15).

FIG. 14 shows a CID mass spectrum of metabolite M1a (m/z 387.10).

FIG. 15 shows a CID mass spectrum of metabolite M1b (m/z 464.06).

FIG. 16 shows a CID mass spectrum of metabolite M1 (m/z 371.10).

FIG. 17 shows a CID mass spectrum of metabolite M3 (m/z 784.22).

FIG. 18 shows a CID mass spectrum of metabolite M4a (m/z 653.17).

FIG. 19 shows a CID mass spectrum of metabolite M5a (m/z 653.17).

FIG. 20 shows a CID mass spectrum of metabolite M5b (m/z 174.09).

FIG. 21 shows a CID mass spectrum of metabolite M5 (m/z 288.03).

FIG. 22 shows a CID mass spectrum of metabolite M6a (m/z 637.18).

FIG. 23 shows a CID mass spectrum of metabolite M6 (m/z 477.14).

FIG. 24 shows a CID mass spectrum of metabolite M7 (m/z 447.13).

FIG. 25 shows a CID mass spectrum of metabolite M8 (m/z 477.14).

FIG. 26 shows a CID mass spectrum of metabolite M9a (m/z 385.08).

FIG. 27 shows a CID mass spectrum of metabolite M9 (m/z 302.04).

### DETAILED DESCRIPTION

Compound 1 (i.e., (S)-4-((1-benzylpyrrolidin-3-yl)(methyl)amino)-2-fluoro-5-methyl-N-(thiazol-4-yl)benzenesulfonamide) is a sodium channel blocker that is selective for Nav1.6 (see e.g., U.S. Pat. Nos. 10,246,453, 10,662,184, 10,815,229, and 11,299,490, the disclosures of which are each incorporated herein by reference in their entireties).

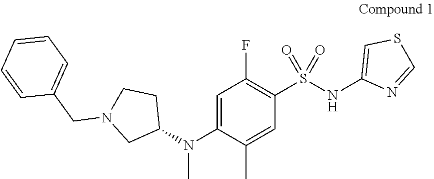 [SMILES: Cc1cc(S(=O)(=O)Nc2cscn2)c(F)cc1N(C)[C@H]1CCN(Cc2ccccc2)C1]

The present application provides deuterated analogs of Compound 1, and pharmaceutically acceptable salts thereof. Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

In some embodiments, the present application provides a compound of Formula I:

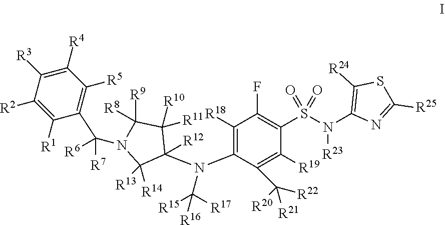

or a pharmaceutically acceptable salt thereof, wherein:
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each independently selected from hydrogen and deuterium; and
wherein at least one R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ is deuterium.

In some embodiments, one to twenty-five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are deuterium, for example, one to twenty, one to eighteen, one to sixteen, one to fourteen, one to twelve, one to ten, one to eight, one to six, one to four, or one to two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are deuterium.

In some embodiments, one to six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are deuterium. In some embodiments, two to six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are deuterium. In some embodiments, two to four of R¹, R², R³, R⁴, R⁵, R⁶, R⁷, R⁸, R⁹, R, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are deuterium. In some embodiments, four to six of R¹, R², R³, R⁴, R⁵, R⁶, R⁷, R, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are deuterium.

In some embodiments, one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ is deuterium. In some embodiments, two of R, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium. In some embodiments, three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium. In some embodiments, four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium. In some embodiments, five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium. In some embodiments, six of R¹, R², R³, R⁴, R⁵, R⁶, R⁷, R, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium. In some embodiments, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

In some embodiments, the compound of Formula I is a compound of Formula II:

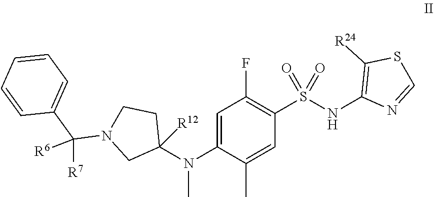

or a pharmaceutically acceptable salt thereof.

In some embodiments, the compound of Formula I is a compound of Formula IIa:

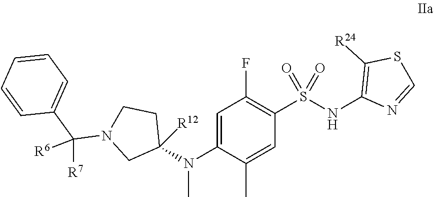

or a pharmaceutically acceptable salt thereof.

In some embodiments, the compound of Formula I is a compound of Formula III:

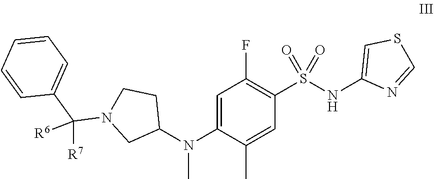

or a pharmaceutically acceptable salt thereof.

In some embodiments, the compound of Formula I is a compound of Formula IIIa:

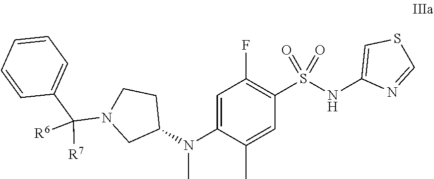

or a pharmaceutically acceptable salt thereof.

In some embodiments, the compound of Formula I is a compound of Formula IV:

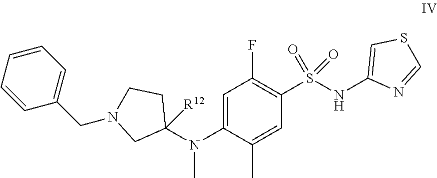

or a pharmaceutically acceptable salt thereof.

In some embodiments, the compound of Formula I is a compound of Formula IVa:

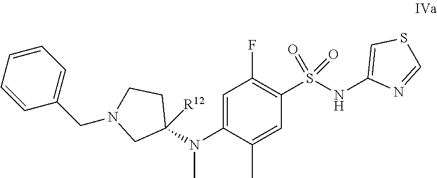

or a pharmaceutically acceptable salt thereof.

In some embodiments, the compound of Formula I is a compound of Formula V:

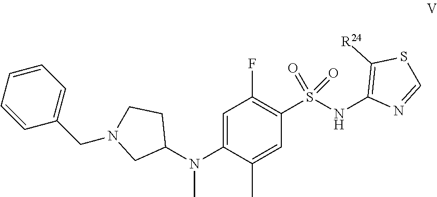

or a pharmaceutically acceptable salt thereof.

In some embodiments, the compound of Formula I is a compound of Formula Va:

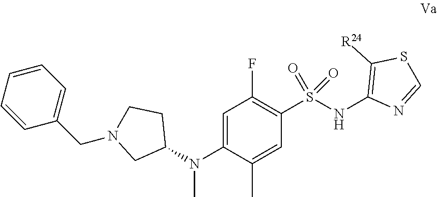

or a pharmaceutically acceptable salt thereof.

In some embodiments, the compound of Formula I is a compound of Formula VI:

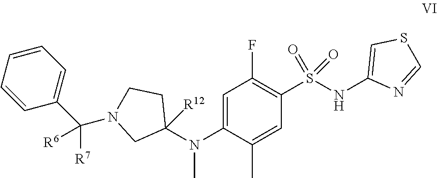

or a pharmaceutically acceptable salt thereof.

In some embodiments, the compound of Formula I is a compound of Formula VIa:

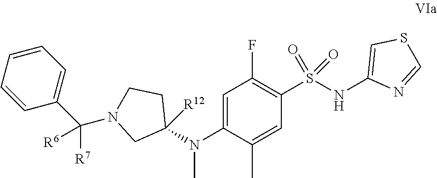

or a pharmaceutically acceptable salt thereof.

In some embodiments, the compound of Formula I is a compound of Formula VII:

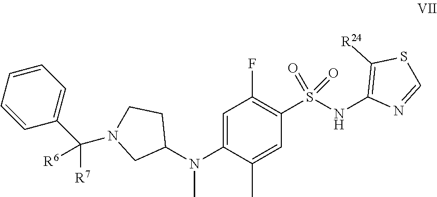

or a pharmaceutically acceptable salt thereof.

In some embodiments, the compound of Formula I is a compound of Formula VIIa:

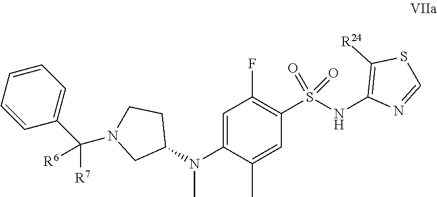

or a pharmaceutically acceptable salt thereof.

In some embodiments, the compound of Formula I is a compound of Formula VIII:

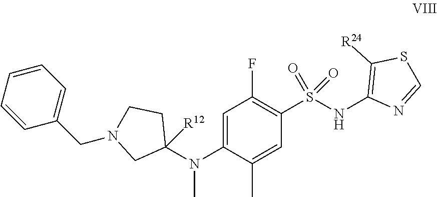

or a pharmaceutically acceptable salt thereof.

In some embodiments, the compound of Formula I is a compound of Formula VIIIa:

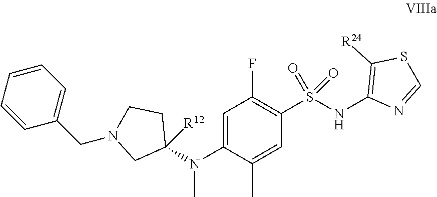

or a pharmaceutically acceptable salt thereof.

In some embodiments, the compound provided herein is selected from:

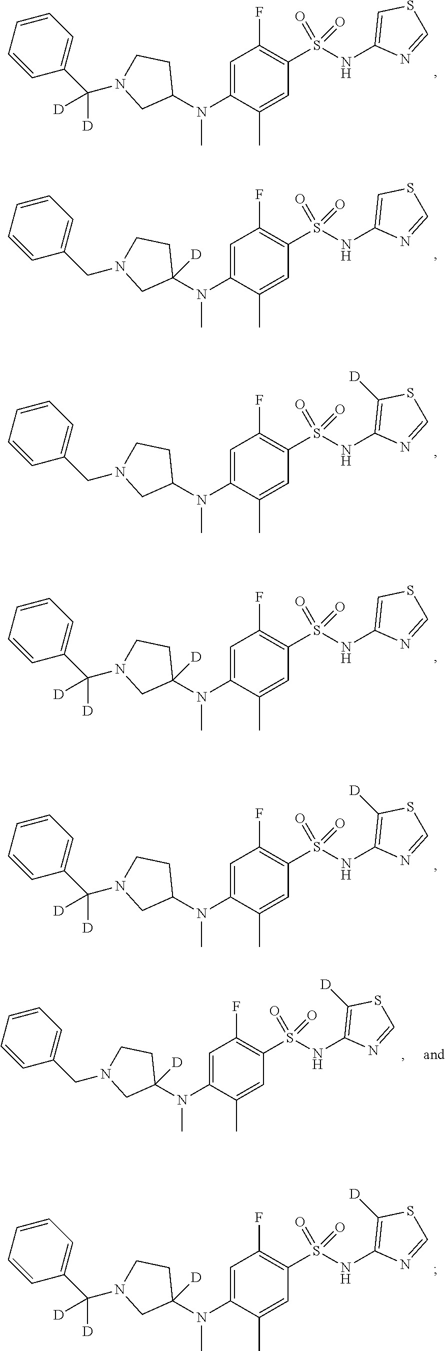

or a pharmaceutically acceptable salt thereof.

In some embodiments, the compound provided herein is selected from:

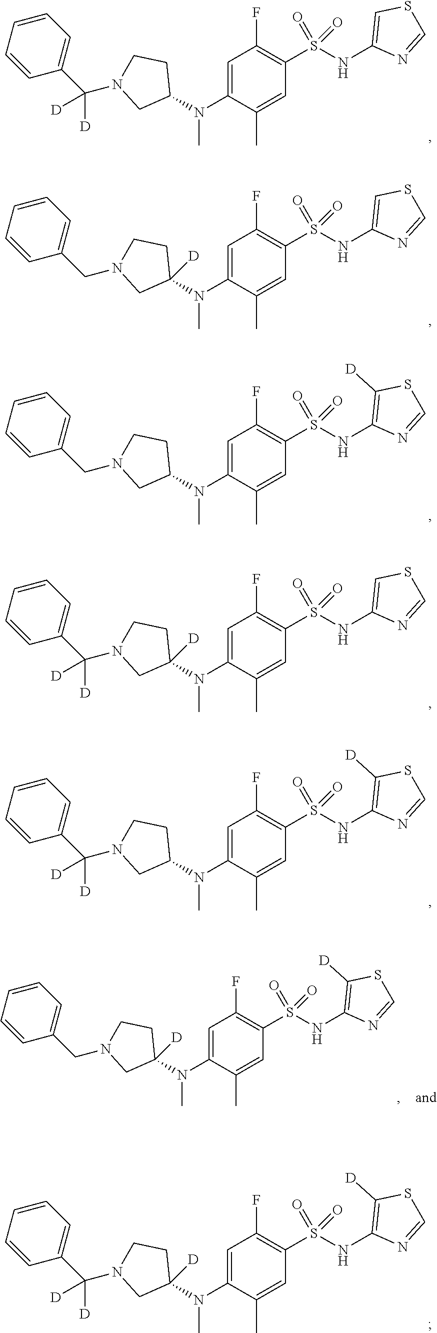

or a pharmaceutically acceptable salt thereof.

The compounds disclosed and described herein allow atoms at each position of the compound independently to have: 1) an isotopic distribution for a chemical element in proportional amounts to those usually found in nature or 2) an isotopic distribution in proportional amounts different to those usually found in nature unless the context clearly dictates otherwise. A particular chemical element has an atomic number defined by the number of protons within the atom's nucleus. Each atomic number identifies a specific element, but not the isotope; an atom of a given element may have a wide range in its number of neutrons. The number of both protons and neutrons in the nucleus is the atom's mass number, and each isotope of a given element has a different mass number. A compound wherein one or more atoms have an isotopic distribution for a chemical element in proportional amounts different to those usually found in nature is commonly referred to as being an isotopically-labeled compound. Each chemical element as represented in a compound structure may include any isotopic distribution of said element. For example, in a compound structure a hydrogen atom may be explicitly disclosed or understood to be present in the compound. At any position of the compound that a hydrogen atom may be present, the hydrogen atom can be an isotopic distribution of hydrogen, including but not limited to protium (H) and deuterium (²H) in proportional amounts to those usually found in nature and in proportional amounts different to those usually found in nature. Thus, reference herein to a compound encompasses all potential isotopic distributions for each atom unless the context clearly dictates otherwise. Examples of isotopes include isotopes of hydrogen, carbon, nitrogen, oxygen, phosphorous, sulfur, fluorine, chlorine, bromine, and iodine. As one of skill in the art would appreciate, any of the compounds as disclosed and described herein may include radioactive isotopes. Accordingly, also contemplated is use of compounds as disclosed and described herein, wherein one or more atoms have an isotopic distribution different to those usually found in nature, such as having ²H or ³H in greater proportion, or ¹¹C, ¹³, or ¹⁴C in greater proportion than found in nature. By way of general example, and without limitation, isotopes of hydrogen include protium (¹H), deuterium (²H), and tritium (³H). Isotopes of carbon include carbon-11 (¹¹C), carbon-12 (¹²C), carbon-13 (¹³C), and carbon-14 (¹⁴C). Isotopes of nitrogen include nitrogen-13 (¹³N), nitrogen-14 (¹⁴N) and nitrogen-15 (¹⁵N). Isotopes of oxygen include oxygen-14 (¹⁴O), oxygen-15 (¹⁵O), oxygen-16 (¹⁶O), oxygen-17 (¹⁷O), and oxygen-18 (¹⁸O). Isotope of fluorine include fluorine-17 (¹⁷F), fluorine-18 (¹⁸F) and fluorine-19 (¹⁹F). Isotopes of phosphorous include phosphorus-31 (³¹P), phosphorus-32 (³²P), phosphorus-33 (³³P), phosphorus-34 (³⁴P), phosphorus-35 (³⁵P) and phosphorus-36 (³⁶P). Isotopes of sulfur include sulfur-32 (³²S), sulfur-33 (³³S), sulfur-34 (³⁴S), sulfur-35 (³⁵S), sulfur-36 (³⁶S) and sulfur-38 (³⁸S). Isotopes of chlorine include chlorine-35 (³⁵Cl), chlorine-36 (³⁶Cl) and chlorine-37 (³⁷Cl). Isotopes of bromine include bromine-75 (⁷⁵Br), bromine-76 (⁷⁶Br), bromine-77 (⁷⁷Br), bromine-79 (⁷⁹Br), bromine-81 (⁸¹Br) and bromine-82 (⁸²Br). Isotopes of iodine include iodine-123 (¹²³I), iodine-124 (¹²⁴I), iodine-125 (¹²⁵I), iodine-131 (¹³¹I) and iodine-135 (¹³⁵I). In some embodiments, atoms at every position of the compound have an isotopic distribution for each chemical element in proportional amounts to those usually found in nature. In some embodiments, an atom in one position of the compound has an isotopic distribution for a chemical element in proportional amounts different to those usually found in nature (remainder atoms having an isotopic distribution for a chemical element in proportional amounts to those usually found in nature). In some embodiments, atoms in at least two positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least three positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least four positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least five positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least six positions of the compound independently have an isotopic distribution for a chemical element in proportional amounts different to those usually found in nature (remainder atoms having an isotopic distribution for a chemical element in proportional amounts to those usually found in nature).

Certain compounds, for example those having incorporated radioactive isotopes such as ³H and ¹⁴C, are also useful in drug or substrate tissue distribution assays. Tritium (³H) and carbon-14 (¹⁴C) isotopes are particularly preferred for their ease of preparation and detectability. Compounds with isotopes such as deuterium (²H) in proportional amounts greater than usually found in nature may afford certain therapeutic advantages resulting from greater metabolic stability, such as, for example, increased in vivo half-life or reduced dosage requirements. Isotopically-labeled compounds can generally be prepared by performing procedures routinely practiced in the chemical art. Methods are readily available to measure such isotope perturbations or enrichments, such as, mass spectrometry, and for isotopes that are radio-isotopes additional methods are available, such as, radio-detectors used in connection with HPLC or GC.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, protium (H), deuterium (²H), tritium (H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound of the invention contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can include ²H as the major isotopic form, as example, or any carbon include be 13C as the major isotopic form, as example, or any nitrogen can include ¹⁵N as the major isotopic form, as example, and any oxygen can include ¹⁸O as the major isotopic form, as example. In certain embodiments, an “isotopic variant” of a compound contains an unnatural proportion of deuterium (²H).

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

The present disclosure further provides synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

### Methods of Use

The compounds provided herein can modulate (e.g., inhibit), ion flux through a voltage-dependent sodium channel (e.g., Nav1.6) in a mammal (e.g., a human). Any such modulation, whether it be partial or complete inhibition or prevention of ion flux, is sometimes referred to herein as “blocking” and corresponding compounds as “blockers” or “inhibitors”. In general, the compounds of the invention modulate the activity of a voltage-gated sodium channel downwards by inhibiting the voltage-dependent activity of the sodium channel, and/or reduce or prevent sodium ion flux across a cell membrane by preventing sodium channel activity such as ion flux.

The compounds provided herein inhibit the ion flux through a voltage-dependent sodium channel (e.g., Nav1.6). The compounds of the invention are state or frequency dependent modifiers of the sodium channel, having a low affinity for the rested/closed state and a high affinity for the inactivated state. These compounds are likely to interact with overlapping sites located in the inner cavity of the sodium conducting pore of the channel similar to that described for other state-dependent sodium channel blockers. These compounds may also be likely to interact with sites outside of the inner cavity and have allosteric effects on sodium ion conduction through the channel pore.

Accordingly, the present invention provides methods for the prevention or treatment of a disease or disorder described herein comprising administering to a subject a compound of Formula I (e.g., a therapeutically effective amount of a compound of Formula I), or a pharmaceutically acceptable salt thereof.

In some embodiments, the compound is administered in an amount of between about 1 mg and about 1000 mg per day, for example, between about 5 mg and about 500 mg per day, about 25 mg and about 400 mg per day, or about 50 mg and about 200 mg per day.

In some embodiments, the compounds provided herein (e.g., compounds of Formula I), or salts thereof, may be useful as standards or controls in in vitro or in vivo assays in determining the efficacy of test compounds in modulating voltage-dependent sodium channels.

In some embodiments, the present application provides a method of treating a disease or a condition associated with Nav1.6 activity in a mammal, wherein the disease or condition is epilepsy and/or epileptic seizure disorder, and wherein the method comprises administering to the mammal in need thereof a therapeutically effective amount of a compound provided herein, or a pharmaceutically acceptable salt thereof.

In some embodiments, the epilepsy or epileptic seizure disorder is photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH 19 epilepsy, progressive myoclonic epilepsies, Rasmussen's syndrome, ring chromosome syndrome, reflex epilepsies, temporal lobe epilepsy, Lafera progressive myoclonus epilepsy, neurocutaneous syndromes, tuberous sclerosis complex, early infantile epileptic encephalopathy, early onset epileptic encephalopathy, SCN8A developmental and epileptic encephalopathy (SCN8A-DEE), focal onset seizure including adult focal onset seizure, generalized epilepsy with febrile seizures, Rett syndrome, multiple sclerosis, Alzheimer's disease, autism, ataxia, hypotonia, or paroxysmal dyskinesia.

In some embodiments, the epilepsy or epileptic seizure disorder is selected from Dravet syndrome, infantile spasms/West's syndrome, temporal lobe epilepsy, Lennox-Gastaut syndrome (LGS), generalized epilepsy with febrile seizures+ and early infantile epileptic encephalopathy.

In some embodiments, the epilepsy or epileptic seizure disorder is SCN8A developmental and epileptic encephalopathy (SCN8A-DEE) or adult focal onset seizure.

In some embodiments, the epilepsy or epileptic seizure disorder is SCN8A developmental and epileptic encephalopathy (SCN8A-DEE).

In some embodiments, the epilepsy or epileptic seizure disorder is adult focal onset seizure.

In some embodiments, the present application provides a method of decreasing ion flux through Nav1.6 in a mammalian cell, wherein the method comprises contacting the cell with a compound provided herein, or a pharmaceutically acceptable salt thereof.

In some embodiments, the present application provides a method of selectively inhibiting a first voltage-gated sodium channel over a second voltage-gated sodium channel in a mammal, wherein the method comprises administering to the mammal a compound provided herein, or a pharmaceutically acceptable salt thereof.

In some embodiments, the first voltage-gated sodium channel is Nav1.6. In some embodiments, the first voltage-gated sodium channel is Nav1.6 and the second voltage-gated sodium channel is Nav1.5. In some embodiments, the first voltage-gated sodium channel is Nav1.6 and the second voltage-gated sodium channel is Nav1.1.

The general value of compounds in inhibiting the Nav1.6 ion flux can be determined, for example, using assays described in U.S. Pat. Nos. 10,246,453, 10,662,184, 10,815,229, and 11,299,490, the disclosures of which are each incorporated herein by reference in their entireties). Alternatively, the general value of the compounds in treating conditions and diseases in humans may be established in industry standard animal models for demonstrating the efficacy of compounds in treating epilepsy and/or epileptic seizure disorder. Animal models of human epileptic conditions have been developed that result in reproducible sensory deficits over a sustained period of time that can be evaluated by sensory testing.

In some embodiments, the compounds provided herein may be metabolized by one or more cytochrome P₄₅₀ isoforms. Examples of cytochrome P₄₅₀ isoforms in a subject (e.g., a mammalian subject), include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F1, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

As used herein, the term “contacting” refers to the bringing together of indicated moieties in an in vitro system or an in vivo system.

As used herein, the term “patient” or “subject” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, subject, or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In some embodiments, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in a subject who is experiencing or displaying the pathology or symptomatology of the disease, condition or disorder (i.e., arresting further development of the pathology and/or symptomatology) or ameliorating the disease; for example, ameliorating a disease, condition or disorder in a subject who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in a subject who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Pharmaceutical Formulations

The present application further provides pharmaceutical compositions containing the compounds provided herein. In some embodiments, the present application provides a composition (e.g., a pharmaceutical composition) comprising a compound provided herein (e.g., a compound of Formula I), or a pharmaceutically acceptable salt thereof, in a pharmaceutically acceptable carrier, excipient or diluent and in an amount effective to modulate, preferably inhibit, ion flux through a voltage-dependent sodium channel to treat sodium channel mediated diseases, such as epilepsy and/or epileptic seizure disorder, when administered to an subject (e.g., a human).

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrasternal injection, or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; see e.g., The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions provided herein further comprise a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in Remington's Pharmaceutical Sciences (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The most suitable route will depend on the nature and severity of the condition being treated. Those skilled in the art are also familiar with determining administration methods (e.g., oral, intravenous, inhalation, sub-cutaneous, rectal, etc.), dosage forms, suitable pharmaceutical excipients and other matters relevant to the delivery of the compounds to a subject in need thereof.

### Combination Therapies

The compounds of the invention may be usefully combined with one or more other compounds of the invention or one or more other therapeutic agent or as any combination thereof, in the treatment of diseases and conditions associated with voltage-gated sodium channel activity. For example, a compound of the invention may be administered simultaneously, sequentially or separately in combination with other therapeutic agents, including, but not limited to:
opiates analgesics, e.g., morphine, heroin, cocaine, oxymorphine, levorphanol, levallorphan, oxycodone, codeine, dihydrocodeine, propoxyphene, nalmefene, fentanyl, hydrocodone, hydromorphone, meripidine, methadone, nalorphine, naloxone, naltrexone, buprenorphine, butorphanol, nalbuphine and pentazocine;
non-opiate analgesics, e.g., acetaminophen, salicylates (e.g., aspirin);
nonsteroidal anti-inflammatory drugs (NSAIDs), e.g., ibuprofen, naproxen, fenoprofen, ketoprofen, celecoxib, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac;
anticonvulsants, e.g., carbamazepine, oxcarbazepine, lamotrigine, valproate, topiramate, gabapentin and pregabalin;
antidepressants such as tricyclic antidepressants, e.g., amitriptyline, clomipramine, despramine, imipramine and nortriptyline;
COX-2 selective inhibitors, e.g., celecoxib, rofecoxib, parecoxib, valdecoxib, deracoxib, etoricoxib, and lumiracoxib;
alpha-adrenergics, e.g., doxazosin, tamsulosin, clonidine, guanfacine, dexmetatomidine, modafinil, and 4-amino-6,7-dimethoxy-2-(5-methane sulfonamido-1,2,3,4-tetrahydroisoquinol-2-yl)-5-(2-pyridyl) quinazoline;
barbiturate sedatives, e.g., amobarbital, aprobarbital, butabarbital, butabital, mephobarbital, metharbital, methohexital, pentobarbital, phenobartital, secobarbital, talbutal, theamylal and thiopental;
tachykinin (NK) antagonist, particularly an NK-3, NK-2 or NK-1 antagonist, e.g., (αR, 9R)-7-[3,5-bis(trifluoromethyl)benzyl)]-8,9,10,11-tetrahydro-9-methyl-5-(4-methylphenyl)-7H-[1,4]diazocino[2,1-g][1,7]-naphthyridine-6-13-dione (TAK-637), 5-[[2R,3S)-2-[(1R)-1-[3,5-bis(trifluoromethylphenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy5-(trifluoromethoxy)phenyl]-methylamino]-2-phenylpiperidine (2S,3S);
coal-tar analgesics, in particular paracetamol;
serotonin reuptake inhibitors, e.g., paroxetine, sertraline, norfluoxetine (fluoxetine desmethyl metabolite), metabolite demethylsertraline, ′3 fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine, trazodone and fluoxetine;
noradrenaline (norepinephrine) reuptake inhibitors, e.g., maprotiline, lofepramine, mirtazepine, oxaprotiline, fezolamine, tomoxetine, mianserin, buproprion, buproprion metabolite hydroxybuproprion, nomifensine and viloxazine (Vivalan®)), especially a selective noradrenaline reuptake inhibitor such as reboxetine, in particular (S,S)-reboxetine, and venlafaxine duloxetine neuroleptics sedative/anxiolytics;
dual serotonin-noradrenaline reuptake inhibitors, such as venlafaxine, venlafaxine metabolite O-desmethylvenlafaxine, clomipramine, clomipramine metabolite desmethylclomipramine, duloxetine, milnacipran and imipramine;
acetylcholinesterase inhibitors such as donepezil;
5-HT₃ antagonists such as ondansetron;
metabotropic glutamate receptor (mGluR) antagonists;
local anaesthetic such as mexiletine and lidocaine;
corticosteroid such as dexamethasone;
antiarrhythimics, e.g., mexiletine and phenytoin;
muscarinic antagonists, e.g., tolterodine, propiverine, tropsium chloride, darifenacin, solifenacin, temiverine and ipratropium;
cannabinoids;
vanilloid receptor agonists (e.g., resinferatoxin) or antagonists (e.g., capsazepine);
sedatives, e.g., glutethimide, meprobamate, methaqualone, and dichloralphenazone;
anxiolytics such as benzodiazepines,
antidepressants such as mirtazapine,
topical agents (e.g., lidocaine, capsacin and resiniferotoxin);
muscle relaxants such as benzodiazepines, baclofen, carisoprodol, chlorzoxazone, cyclobenzaprine, methocarbamol and orphrenadine;
anti-histamines or H1 antagonists;
NMDA receptor antagonists;
5-HT receptor agonists/antagonists;
PDEV inhibitors;
Tramadol®;
cholinergic (nicotinic) analgesics;
alpha-2-delta ligands;
prostaglandin E2 subtype antagonists;
leukotriene B4 antagonists;
5-lipoxygenase inhibitors; and
5-HT₃ antagonists.

As used herein, “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Kits

The present invention also provides kits that contain a pharmaceutical composition which includes one or more compounds of the invention. The kit also includes instructions for the use of the pharmaceutical composition for inhibiting the activity of voltage-gated sodium channels, preferably Nav1.6, for the treatment of epilepsy, as well as other utilities as disclosed herein. In some embodiments, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. Metabolite Identification of Compound 1 in Mouse, Rat, Dog, Monkey, and Human Hepatocytes

The purpose of this study was to identify metabolites of Compound 1 in CD-1 mouse, SD rat, Beagle dog, Cynomolgus monkey, and human hepatocytes after incubation for 120 min using LC-UV-MSⁿ (n=1-2) and to propose metabolic pathways. MetaboLynx software was employed for post-acquisition data processing and the structures of metabolites were elucidated based on characteristics of the MS and MS² data.

### Sample Generation and Processing

Cryopreserved hepatocytes: 1.0×10⁶ cells/mL
Test Compound: Compound 1
Test Compound Concentration: 10 μM
Incubation Medium: William's E Medium
Total Incubation Volume: 200 μL×2
Incubation Temperature: 37° C. in 5% CO₂/95% humidity
Incubation Time: 0, 120 min
Sample Processing: Precipitated with 2-fold volume of acetonitrile containing 0.1% formic acid and centrifuged. The supernatant was then dried with N₂ gas at room temperature. The residue was reconstituted with 200 μL of 10% acetonitrile/0.1% formic acid. 15 μL was injected into the LC-MS system for analysis

### Liquid Chromatography-Mass Spectrometry

HPLC System: ACQUITY UPLC
Column: ACQUITY UPLC HSS T3 1.8 μm 2.1×100 mm column
Column Temperature: 40° C.
Autosampler Temperature: 4° C.
Mobile Phase A: 0.1% formic acid in H₂O
Mobile Phase B: 0.1% formic acid in CH₃CN

### Gradient Time Program:

| | Total Time | Flow Rate | | |
|---|---|---|---|---|
| Step | (min) | (μL/min) | A (%) | B (%) |
| 0 | 0.00 | 500 | 90 | 10 |
| 1 | 1.00 | 500 | 90 | 10 |
| 2 | 5.00 | 500 | 80 | 20 |
| 3 | 10.00 | 500 | 70 | 30 |
| 4 | 12.00 | 500 | 45 | 55 |
| 5 | 14.00 | 500 | 10 | 90 |
| 6 | 16.00 | 500 | 10 | 90 |
| 7 | 18.00 | 500 | 90 | 10 |

Mass System: Xevo G2 QTOF
Ionization Mode: ES⁺
Capillary: 2.6 kV
Sampling Cone: 35.0 V
Extraction Cone: 4.0 V
Source Temperature: 100° C.
Desolvation Temperature: 400° C.
Cone Gas Flow: 800 L/h
CE: 23-24 eV
Scan Mode: MS^{E}

Under these experimental conditions, fourteen metabolites were detected and assigned as listed below.
M1a: Oxygenation and N-dealkylation (MW=386.09, P+O—C₇H₆)
M1b: Demethylation, glucuronidation and N-dealkylation (MW=463.05, P—CH₂+C₆H₈O₆—C₁₁H₁₃N)
M1: N-dealkylation (MW=370.09, P— C₇H₆)
M3a: Oxygenation and glutathione conjugation (MW=783.22, P+O+C₁₀H₁₇N₃O₆S)
M4a and M5a: Oxygenation and glucuronidation (MW=652.17, P+O+C₆H₈O₆)
M5b: Oxygenation, di-dehydrogenation, and dealkylation (MW=173.08, P+O-4H—C₁₁H₁₀N₃O₂FS₂)
M5: Demethylation and N-dealkylation (MW=287.02, P—CH₂—C₁₁H₁₃N)
M6a: Glucuronidation (MW=636.17, P+C₆H₈O₆)
M6 and M8: Oxygenation (MW=476.14, P+O)
M7: Demethylation (MW=446.12, P—CH₂)
M9a: Oxygenation, dehydrogenation, N-dealkylation (MW=384.07, P+O—C₇H₆)
M9: Dealkylation (MW=301.04, P—C₁₁H₁₃N)

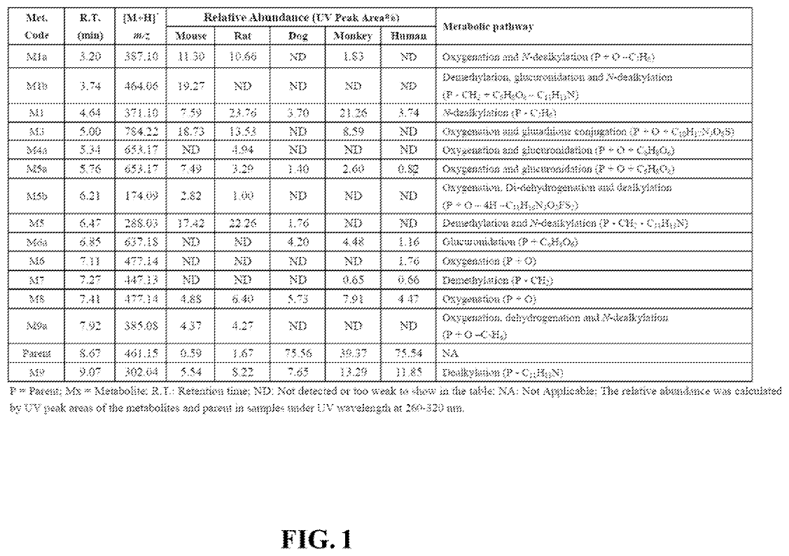

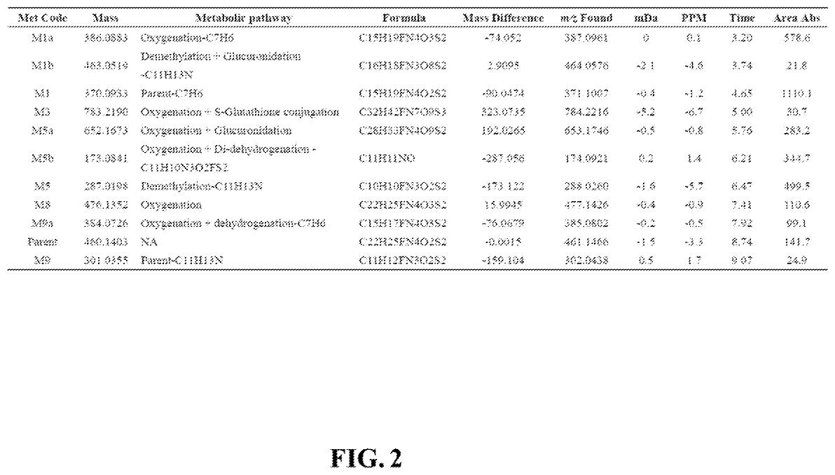

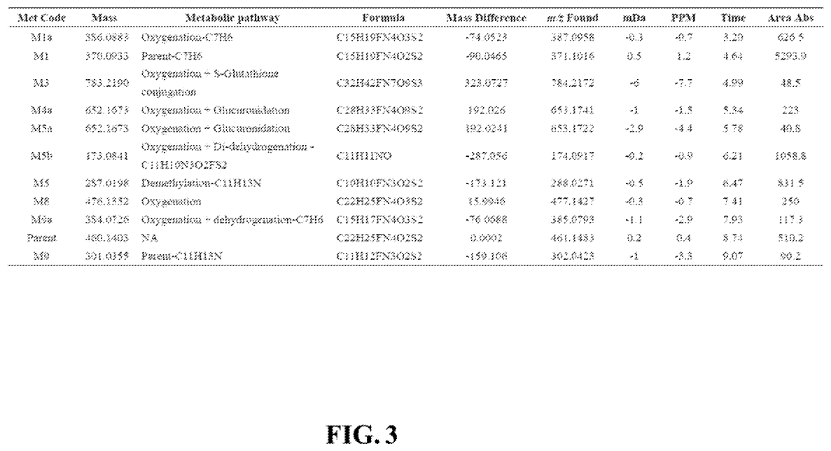

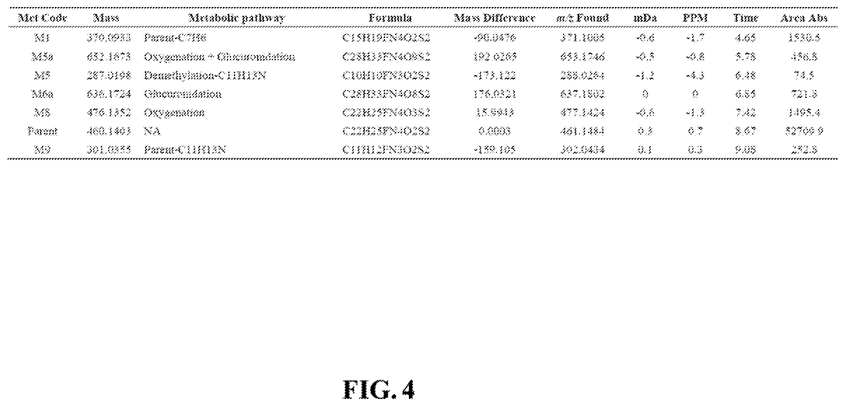

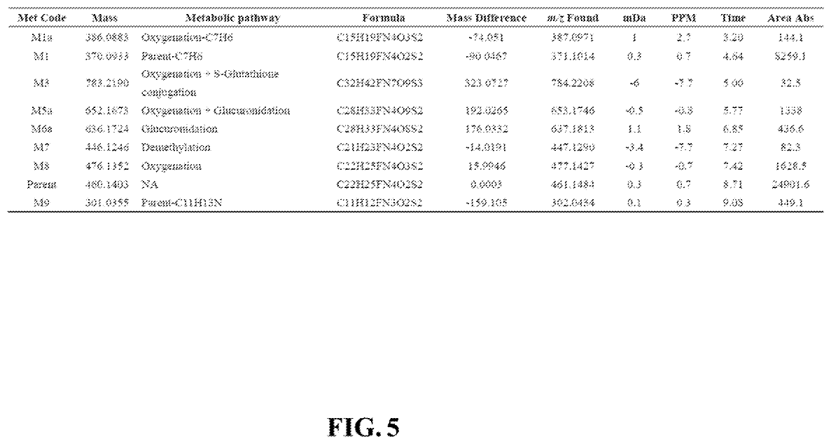

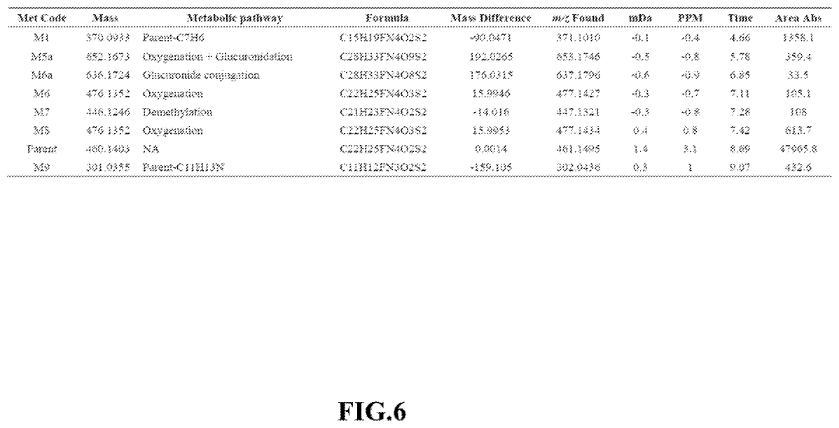

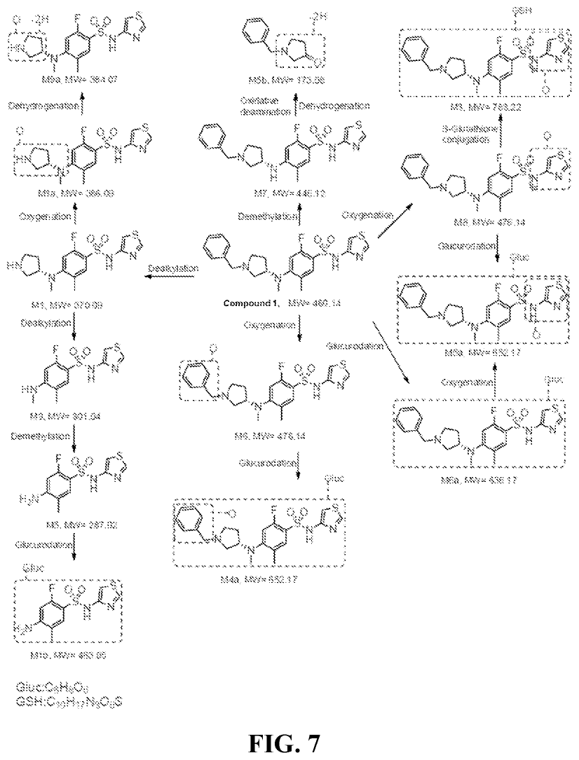

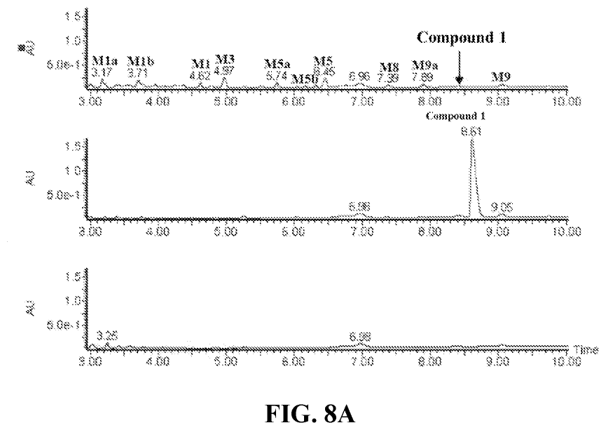

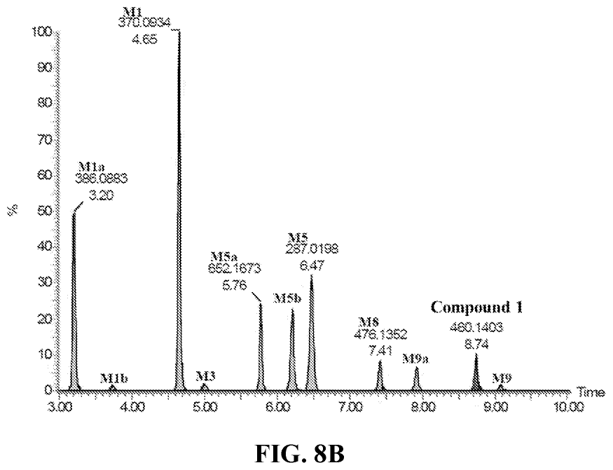

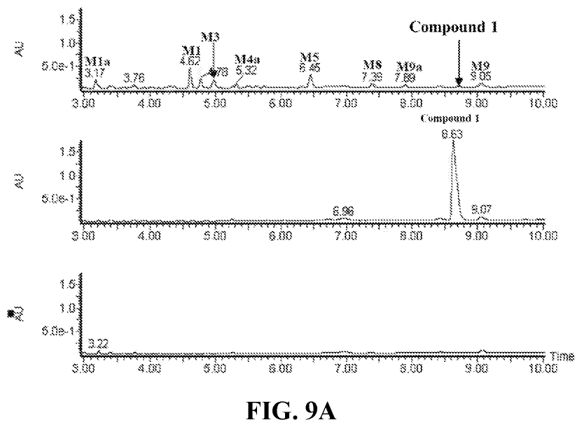

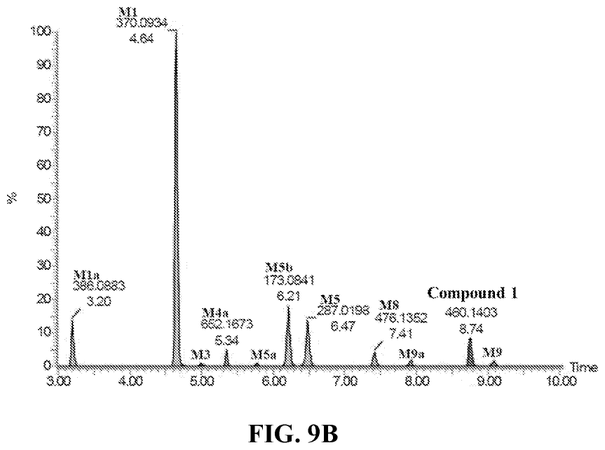

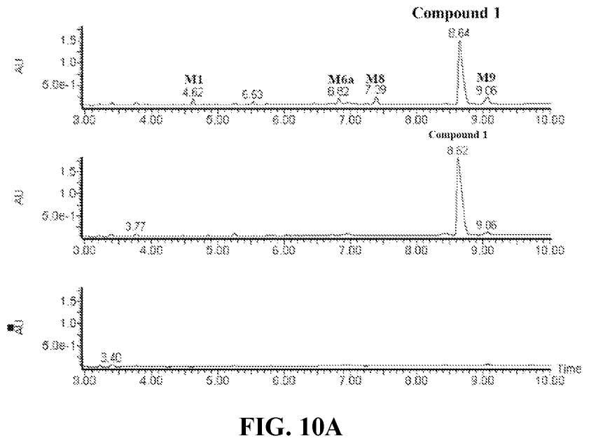

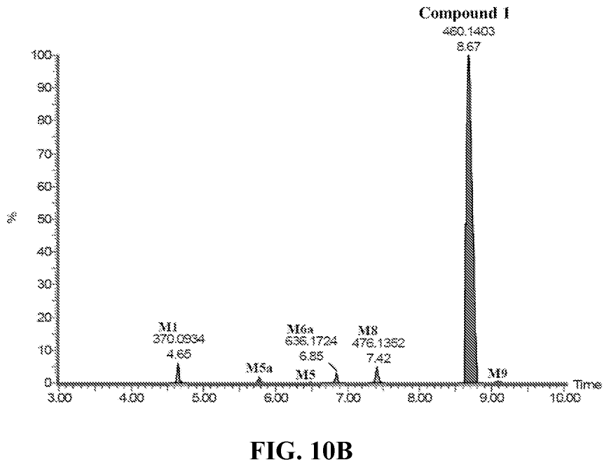

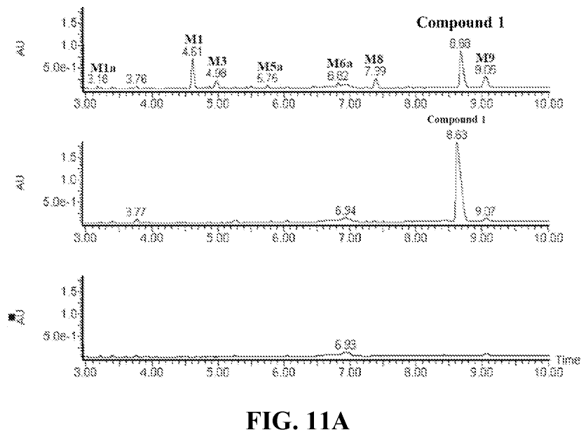

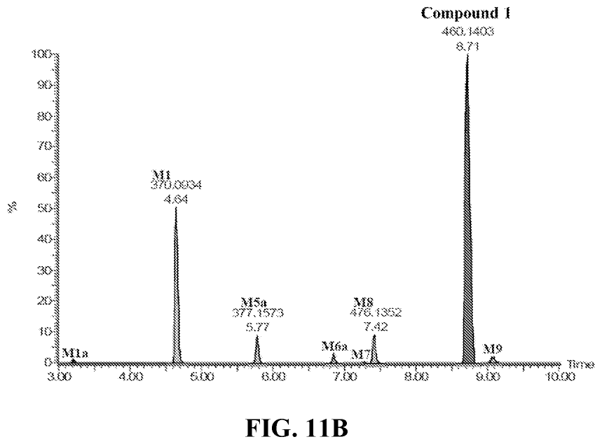

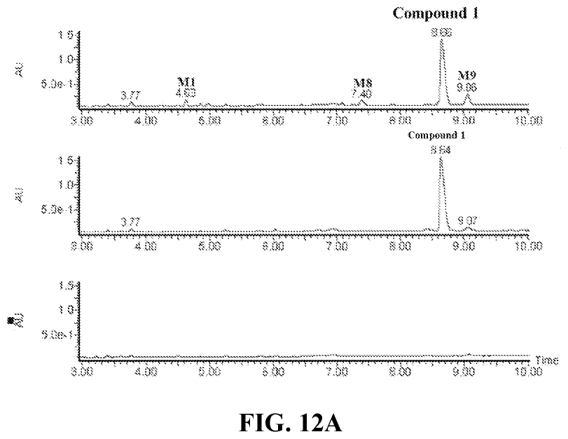

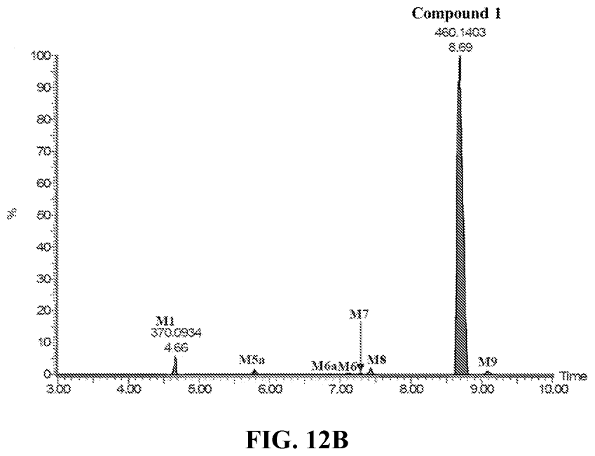

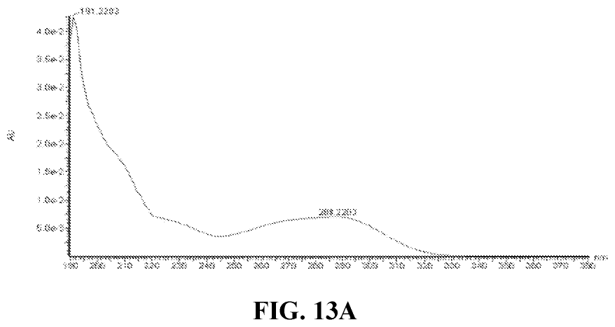

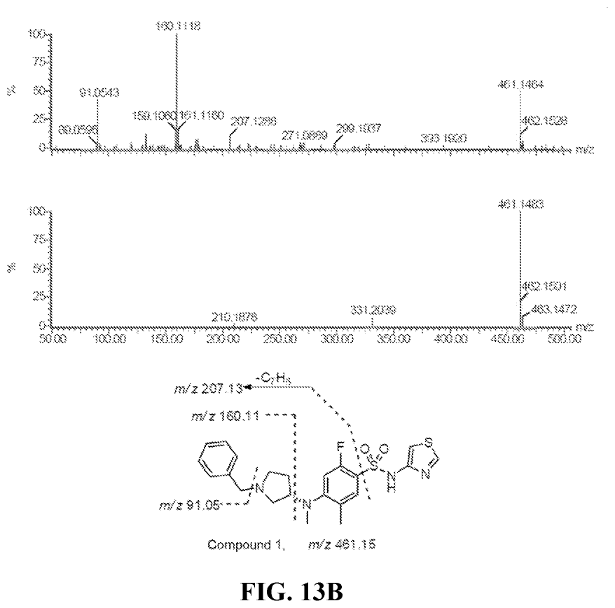

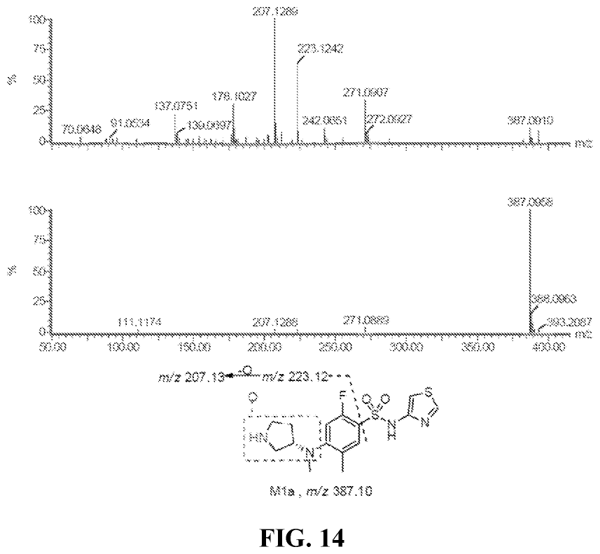

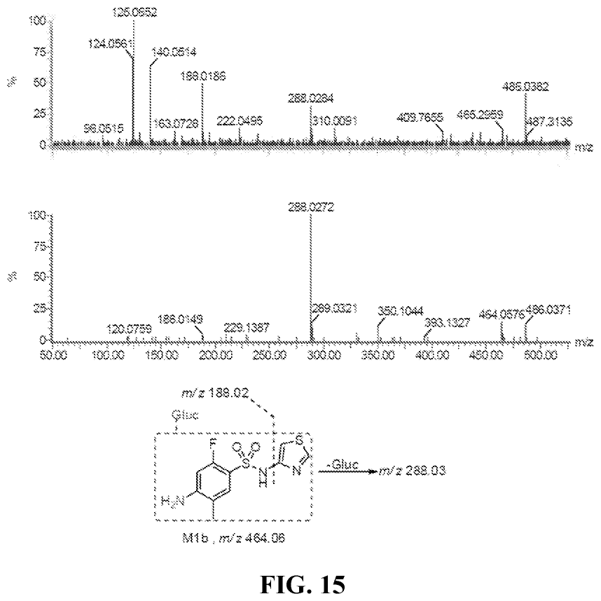

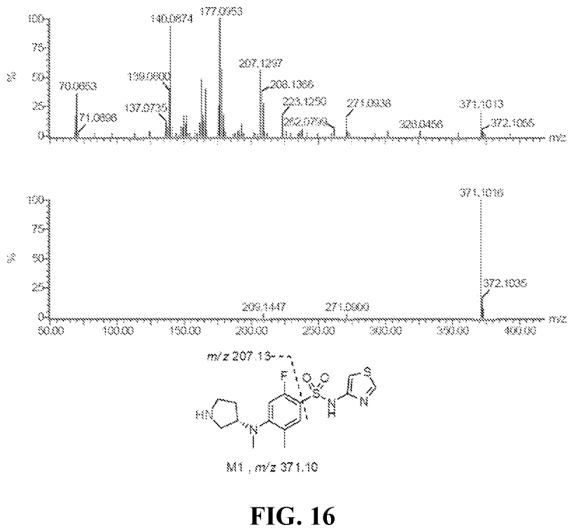

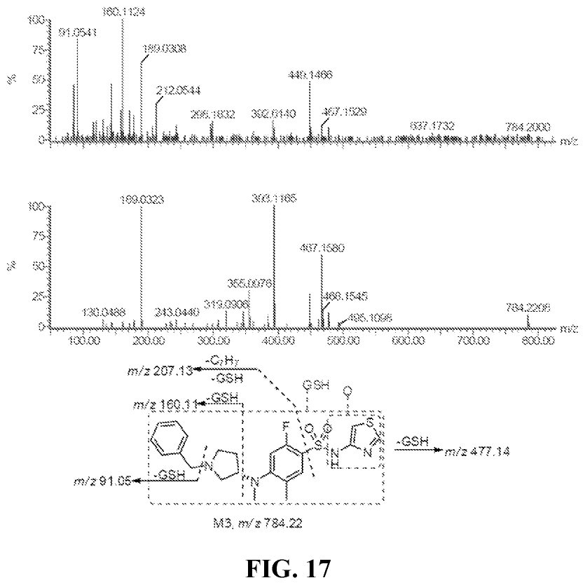

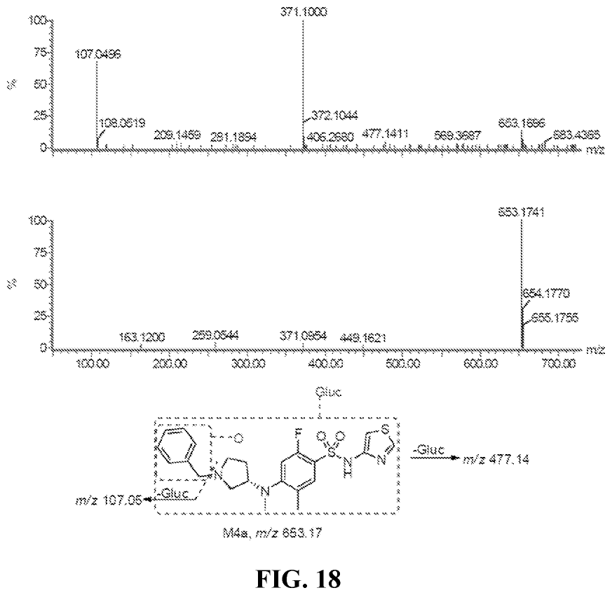

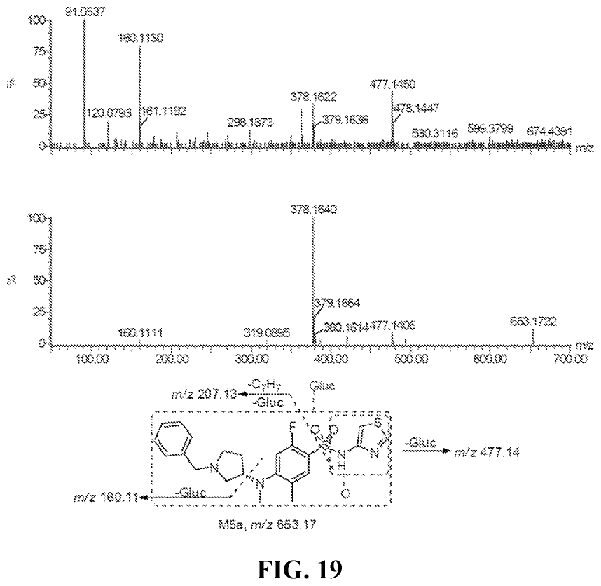

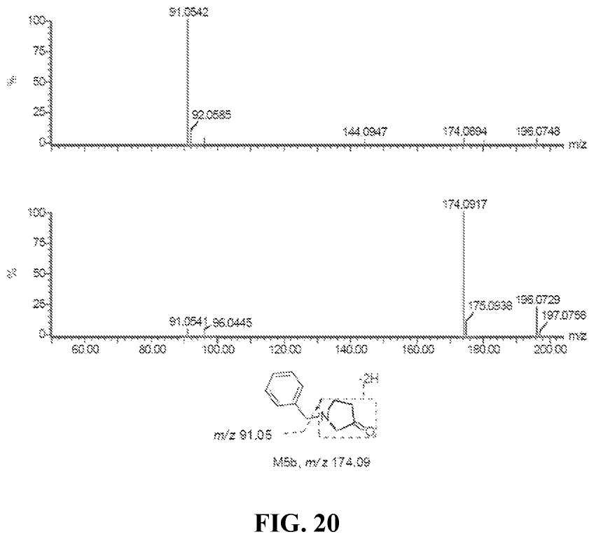

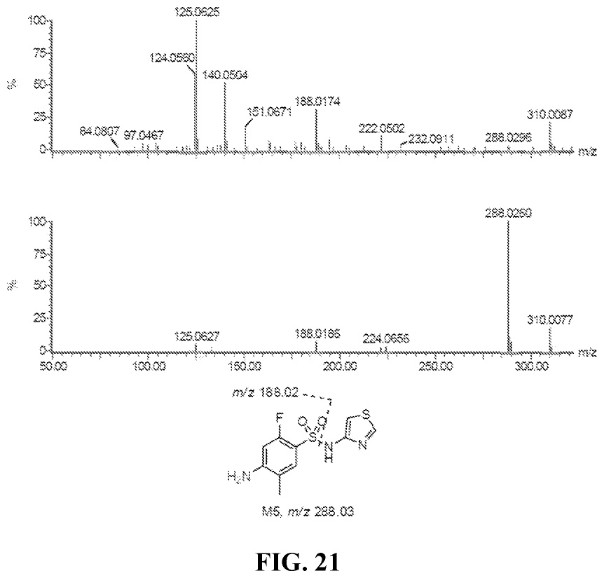

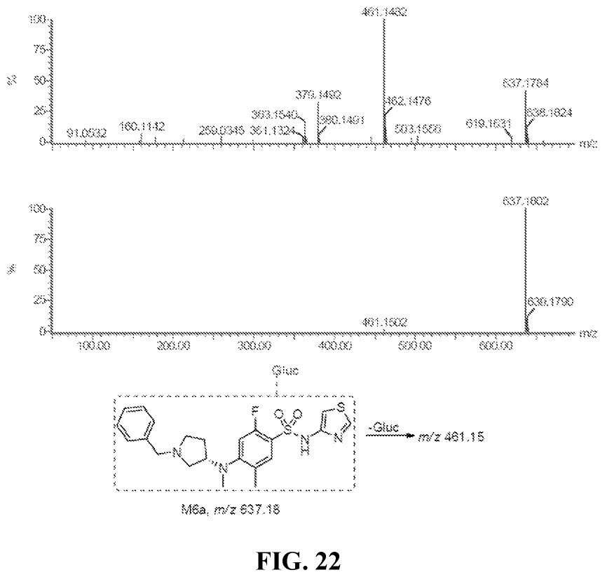

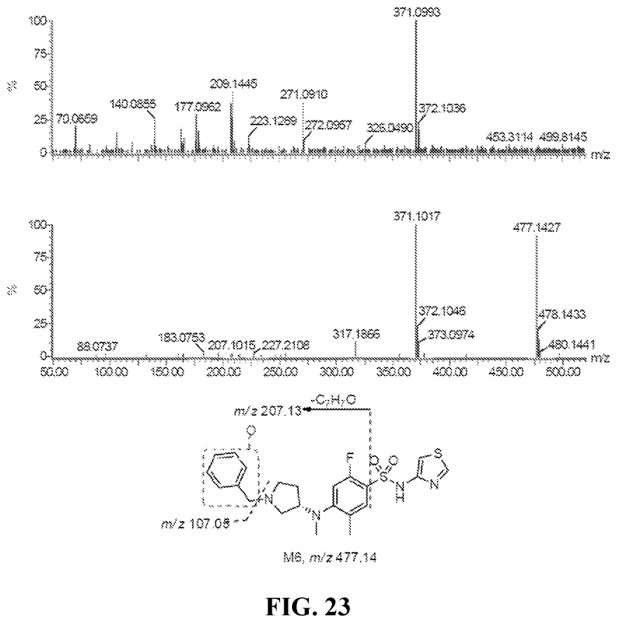

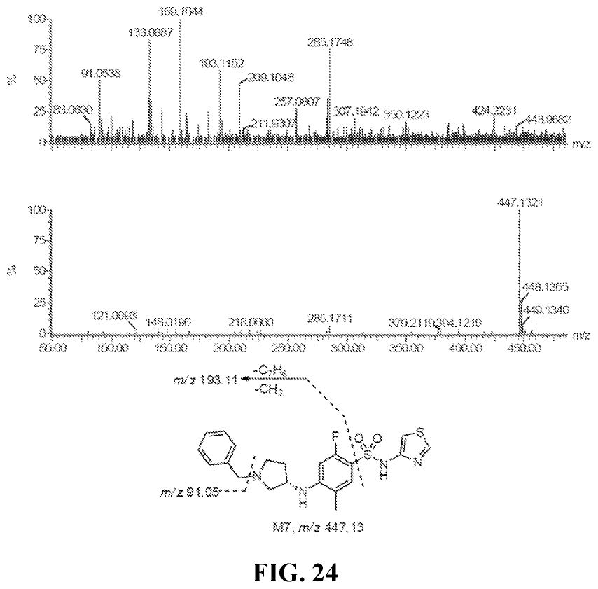

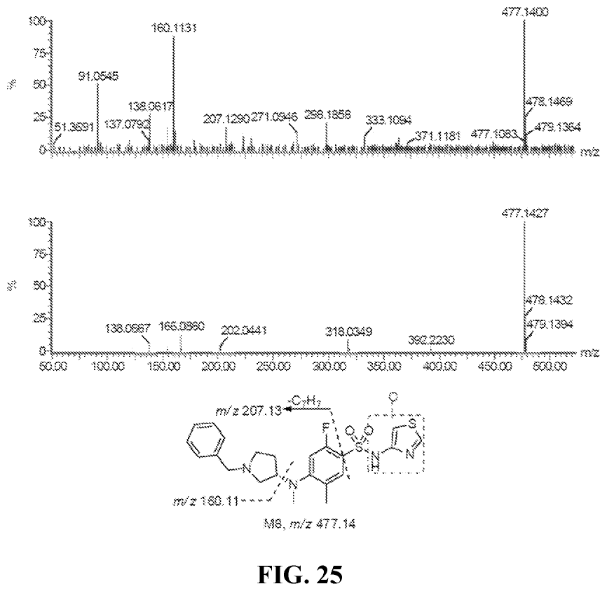

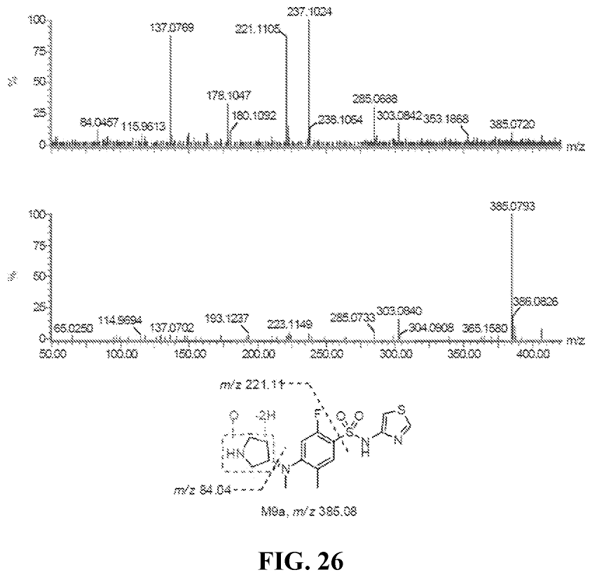

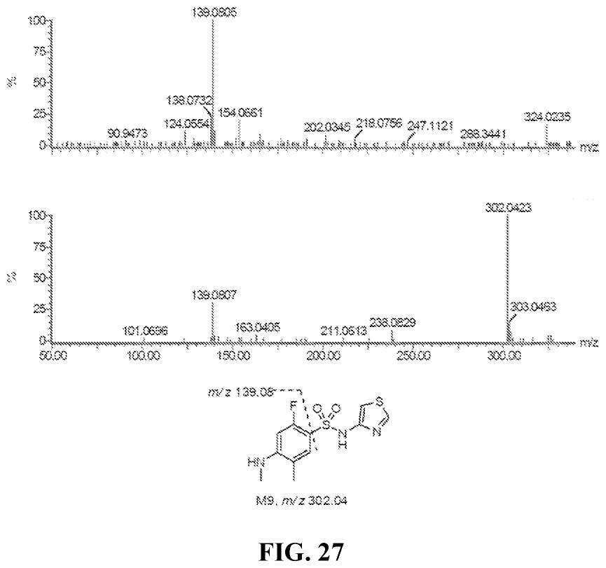

Metabolites were further confirmed and structurally characterized based on their retention times, accurate masses, and characteristics of their fragment ions. The information of the metabolites from mouse, rat, dog, monkey, and human hepatocytes is summarized in FIG. 1 and the related information on LC-MS^{E} scans are shown in FIGS. 2-6. The LC-UV and LC-MS chromatograms in mouse, rat, dog, monkey, and human hepatocytes are depicted in FIGS. 8A-12B. The UV and MS² spectra of Compound 1 are shown in FIGS. 13A-13B, while the MS² spectra of the metabolites are shown in FIGS. 14-27. The tentative structures of the metabolites and the metabolic pathways of Compound 1 are shown in FIG. 7.

The main metabolic pathways of Compound 1 in mouse, rat, dog, monkey, and human hepatocytes are incubation for 120 min were oxygenation, glucuronidation, demethylation, dehydrogenation, dealkylation, and glutathione conjugation.

Based on UV peak area percentage, M1a, M1b, M3, and M5 were considered to be the major metabolites (Peak area percentage>10%) in mouse hepatocytes, which account for 11.30%, 19.27%, 18.73%, and 17.42%, respectively. In rat hepatocytes, M1a, M1, M3, and M5 were determined to be the major metabolites with percentages of 10.66%, 23.76%, 13.53%, and 22.26%, respectively. In dog hepatocytes, all the metabolites were determined to be minor metabolites with percentage less than 10%. In monkey hepatocytes, M1 and M9 were found as the major metabolites at 21.26% and 13.29%, respectively. In human hepatocytes, M9 was detected as the major metabolite, with percentage of 11.85%.

### Example 2. Sodium Influx Assay (In Vitro Assay)

This sodium influx assay employs the use of the cell permeable, sodium sensitive dye ANG2 to quantify sodium ion influx through sodium channels which are maintained in an open state by use of sodium channel modulators. This high throughput sodium influx assay allows for rapid profiling and characterization of sodium channel blockers.

In general, Trex HEK293 cells were stably transfected with an inducible expression vector containing the full-length cDNA coding for the desired human sodium channel α-subunit and with an expression vector containing full length cDNA coding for the β1-subunit. Sodium channel expressing cell lines were induced with tetracycline (1 μg/mL) and plated on 384-well PDL-coated plates at a density of 25K-30K cells/well in culture media (DMEM, containing 10% FBS and 1% L-glutamine). After overnight incubation (37° C., 5% CO₂), culture media was removed and cells were loaded with 5 uM ANG2 dye for 1-1.5 h in Buffer 1 (155 mM NMDG, 5 mM KCl, 2 mM CaCl₂), 1 mM MgCl₂, 10 mM HEPES, 10 mM glucose, adjusted with Tris to pH 7.4). Access dye was removed and cells were incubated with test compounds for 1 hr in buffer 1 containing sodium channel modulator(s) at room temperature. Hamamatsu FDSS μCell was used to perform a 1:1 addition of Na/K challenge buffer (140 mM NaCl, 20 mM HEPES, 1 mM CaCl₂), 15 mM KCl, 1 mM MgCl₂, 10 mM glucose, adjusted with Tris to pH 7.4) and simultaneously read plates at excitation wavelength of 530 nm and emission wavelength set at 558 nm. Percent inhibition of sodium ion influx was calculated for each test compound at each test concentration to determine the IC₅₀ values.

Compound 1, when tested in this model, demonstrated affinities for the inactivated state of Nav1.6, Nav1.5 and Nav1.1 as set forth in the table below.

Inhibition of Na_{V}1.6, Na_{V}1.5 and Na_{V}1.1

| | Flux | Flux | Flux |
|---|---|---|---|
| | Na_{V}1.6 | Na_{V}1.5 | Na_{V}1.1 |
| Compound | IC₅₀ (μM) | IC₅₀ (μM) | IC₅₀ (μM) |
| 1 | 0.272 | 24.339 | 15.599 |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 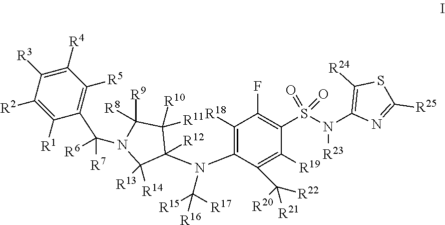
or a pharmaceutically acceptable salt thereof, wherein:
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each independently selected from hydrogen and deuterium; and
wherein at least one R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ is deuterium.

2. The compound of claim 1, or a pharmaceutically acceptable salt thereof, wherein one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ is deuterium.

3. The compound of claim 1, or a pharmaceutically acceptable salt thereof, wherein two of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

4. The compound of claim 1, or a pharmaceutically acceptable salt thereof, wherein three of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

5. The compound of claim 1, or a pharmaceutically acceptable salt thereof, wherein four of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

6. The compound of claim 1, or a pharmaceutically acceptable salt thereof, wherein five of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

7. The compound of claim 1, or a pharmaceutically acceptable salt thereof, wherein six of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

8. The compound of claim 1, or a pharmaceutically acceptable salt thereof, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁵ are each deuterium.

9. The compound of claim 1, which is a compound of Formula II: 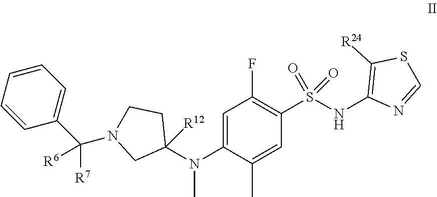
or a pharmaceutically acceptable salt thereof.

10. The compound of claim 1, which is a compound of Formula III: 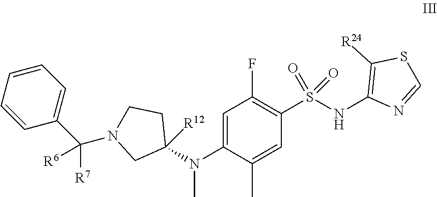
or a pharmaceutically acceptable salt thereof.

11. The compound of claim 1, which is selected from: 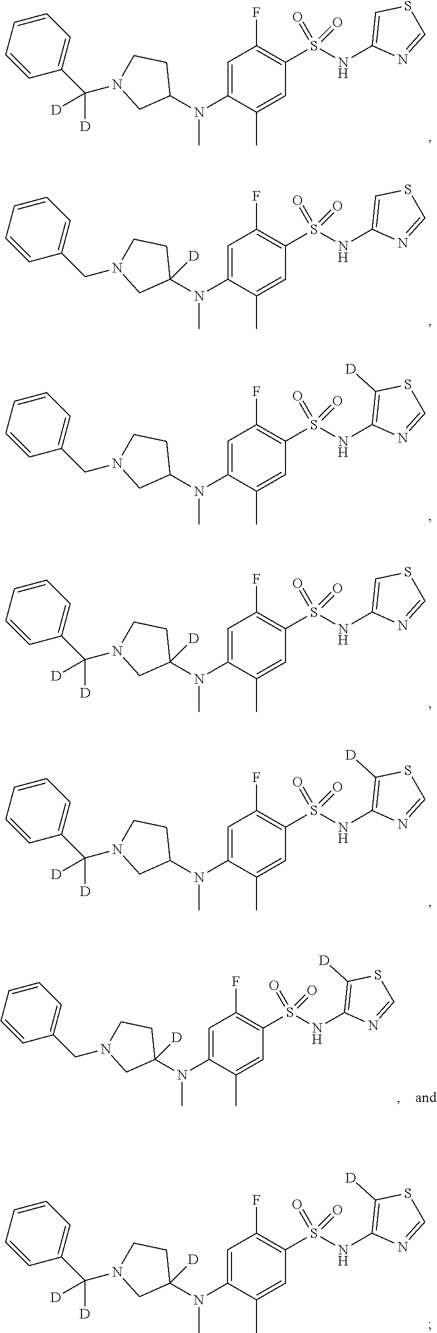
or a pharmaceutically acceptable salt thereof.

12. A pharmaceutical composition, comprising a compound of any one of claims 1 to 11, or a pharmaceutically acceptable salt thereof, and a pharmaceutically acceptable carrier.

13. A method of inhibiting a Nav1.6 channel, comprising contacting a Nav1.6 channel with a compound of any one of claims 1 to 11, or a pharmaceutically acceptable salt thereof.

14. A method of treating a disease or disorder associated with Nav1.6 in a subject, comprising administering to the subject a therapeutically effective amount of a compound of any one of claims 1 to 11, or a pharmaceutically acceptable salt thereof.

15. The method of claim 14, wherein the disease or disorder is epilepsy, epileptic seizure disorder, or a combination thereof.

16. The method of claim 15, wherein the epilepsy or epileptic seizure disorder is photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH 19 epilepsy, progressive myoclonic epilepsies, Rasmussen's syndrome, ring chromosome syndrome, reflex epilepsies, temporal lobe epilepsy, Lafera progressive myoclonus epilepsy, neurocutaneous syndromes, tuberous sclerosis complex, early infantile epileptic encephalopathy, early onset epileptic encephalopathy, SCN8A developmental and epileptic encephalopathy (SCN8A-DEE), focal onset seizure including adult focal onset seizure, generalized epilepsy with febrile seizures, Rett syndrome, multiple sclerosis, Alzheimer's disease, autism, ataxia, hypotonia, or paroxysmal dyskinesia.

17. The method of claim 15, wherein the epilepsy or epileptic seizure disorder is SCN8A developmental and epileptic encephalopathy (SCN8A-DEE).

18. The method of claim 15, wherein the epilepsy or epileptic seizure disorder is adult focal onset seizure.
